SEQUENCE LISTING (3 sequences)

SEQ ID NO: 1 - protein, 1404 aa, Homo sapiens
MAWKTLPIYLLLLLSVFVIQQVSSQDLSSCAGRCGEGYSRDATCNCDYNCQHYMECCPDFKRVCTAELSCKGRCFESFERGRECDCDAQCKKYDKCCPDYESFCAEVHNPTSPPSSKKAPPPSGASQTIKSTTKRSPKPPNKKKTKKVIESEEITEEHSVSENQESSSSSSSSSSSSTIRKIKSSKNSAANRELQKKLKVKDNKKNRTKKKPTPKPPVVDEAGSGLDNGDFKVTTPDTSTTQHNKVSTSPKITTAKPINPRPSLPPNSDTSKETSLTVNKETTVETKETTTTNKQTSTDGKEKTTSAKETQSIEKTSAKDLAPTSKVLAKPTPKAETTTKGPALTTPKEPTPTTPKEPASTTPKEPTPTTIKSAPTTPKEPAPTTTKSAPTTPKEPAPTTTKEPAPTTPKEPAPTTTKEPAPTTTKSAPTTPKEPAPTTPKKPAPTTPKEPAPTTPKEPTPTTPKEPAPTTKEPAPTTPKEPAPTAPKKPAPTTPKEPAPTTPKEPAPTTTKEPSPTTPKEPAPTTTKSAPTTTKEPAPTTTKSAPTTPKEPSPTTTKEPAPTTPKEPAPTTPKKPAPTTPKEPAPTTPKEPAPTTTKKPAPTTPKEPAPTTPKETAPTTPKKLTPTTPEKLAPTTPEKPAPTTPEELAPTTPEEPTPTTPEEPAPTTPKAAAPNTPKEPAPTTPKEPAPTTPKEPAPTTPKETAPTTPKGTAPTTLKEPAPTTPKKPAPKELAPTTTKEPTSTTCDKPAPTTPKGTAPTTPKEPAPTTPKEPAPTTPKGTAPTTLKEPAPTTPKKPAPKELAPTTTKGPTSTTSDKPAPTTPKETAPTTPKEPAPTTPKKPAPTTPETPPPTTSEVSTPTTTKEPTTIHKSPDESTPELSAEPTPKALENSPKEPGVPTTKTPAATKPEMTTTAKDKTTERDLRTTPETTTAAPKMTKETATTTEKTTESKITATTTQVTSTTTQDTTPFKITTLKTTTLAPKVTTTKKTITTTEIMNKPEETAKPKDRATNSKATTPKPQKPTKAPKKPTSTKKPKTMPRVRKPKTTPTPRKMTSTMPELNPTSRIAEAMLQTTTRPNQTPNSKLVEVNPKSEDAGGAEGETPHMLLRPHVFMPEVTPDMDYLPRVPNQGIIINPMLSDETNICNGKPVDGLTTLRNGTLVAFRGHYFWMLSPFSPPSPARRITEVWGIPSPIDTVFTRCNCEGKTFFFKDSQYWRFTNDIKDAGYPKPIFKGFGGLTGQIVAALSTAKYKNWPESVYFFKRGGSIQQYIYKQEPVQKCPGRRPALNYPVYGETTQVRRRRFERAIGPSQTHTIRIQYSPARLAYQDKGVLHNEVKVSILWRGLPNVVTSAISLPNIRKPDGYDYYAFSKDQYYNIDVPSRTARAITTRSGQTLSKVWYNCP

SEQ ID NO: 2 - DNA, 5041 bp, Homo sapiens
gcggccgcgactattcggtacctgaaaacaacgatggcatggaaaacacttcccatttacctgttgttgctgctgtctgttttcgtgattcagcaagtttcatctcaagatttatcaagctgtgcagggagatgtggggaagggtattctagagatgccacctgcaactgtgattataactgtcaacactacatggagtgctgccctgatttcaagagagtctgcactgcggagctttcctgtaaaggccgctgctttgagtccttcgagagagggagggagtgtgactgcgacgcccaatgtaagaagtatgacaagtgctgtcccgattatgagagtttctgtgcagaagtgcataatcccacatcaccaccatcttcaaagaaagcacctccaccttcaggagcatctcaaaccatcaaatcaacaaccaaacgttcacccaaaccaccaaacaagaagaagactaagaaagttatagaatcagaggaaataacagaagaacattctgtttctgaaaatcaagagtcctcctcctcctcctcctcttcctcttcttcttcaacaatttggaaaatcaagtcttccaaaaattcagctgctaatagagaattacagaagaaactcaaagtaaaagataacaagaagaacagaactaaaaagaaacctacccccaaaccaccagttgtagatgaagctggaagtggattggacaatggtgacttcaaggtcacaactcctgacacgtctaccacccaacacaataaagtcagcacatctcccaagatcacaacagcaaaaccaataaatcccagacccagtcttccacctaattctgatacatctaaagagacgtctttgacagtgaataaagagacaacagttgaaactaaagaaactactacaacaaataaacagacttcaactgatggaaaagagaagactacttccgctaaagagacacaaagtatagagaaaacatctgctaaagatttagcacccacatctaaagtgctggctaaacctacacccaaagctgaaactacaaccaaaggccctgctctcaccactcccaaggagcccacgcccaccactcccaaggagcctgcatctaccacacccaaagagcccacacctaccaccatcaagtctgcacccaccacccccaaggagcctgcacccaccaccaccaagtctgcacccaccactcccaaggagcctgcacccaccaccaccaaggagcctgcacccaccactcccaaggagcctgcacccaccaccaccaaggagcctgcacccaccaccaccaagtctgcacccaccactcccaaggagcctgcacccaccacccccaagaagcctgccccaactacccccaaggagcctgcacccaccactcccaaggagcctacacccaccactcccaaggagcctgcacccaccaccaaggagcctgcacccaccactcccaaagagcctgcacccactgcccccaagaagcctgccccaactacccccaaggagcctgcacccaccactcccaaggagcctgcacccaccaccaccaaggagccttcacccaccactcccaaggagcctgcacccaccaccaccaagtctgcacccaccactaccaaggagcctgcacccaccactaccaagtctgcacccaccactcccaaggagccttcacccaccaccaccaaggagcctgcacccaccactcccaaggagcctgcacccaccacccccaagaagcctgccccaactacccccaaggagcctgcacccaccactcccaaggaacctgcacccaccaccaccaagaagcctgcacccaccgctcccaaagagcctgccccaactacccccaaggagactgcacccaccacccccaagaagctcacgcccaccacccccgagaagctcgcacccaccacccctgagaagcccgcacccaccacccctgaggagctcgcacccaccacccctgaggagcccacacccaccacccctgaggagcctgctcccaccactcccaaggcagcggctcccaacacccctaaggagcctgctccaactacccctaaggagcctgctccaactacccctaaggagcctgctccaactacccctaaggagactgctccaactacccctaaagggactgctccaactaccctcaaggaacctgcacccactactcccaagaagcctgcccccaaggagcttgcacccaccaccaccaaggagcccacatccaccacctctgacaagcccgctccaactacccctaaggggactgctccaactacccctaaggagcctgctccaactacccctaaggagcctgctccaactacccctaaggggactgctccaactaccctcaaggaacctgcacccactactcccaagaagcctgcccccaaggagcttgcacccaccaccaccaaggggcccacatccaccacctctgacaagcctgctccaactacacctaaggagactgctccaactacccccaaggagcctgcacccactacccccaagaagcctgctccaactactcctgagacacctcctccaaccacttcagaggtctctactccaactaccaccaaggagcctaccactatccacaaaagccctgatgaatcaactcctgagctttctgcagaacccacaccaaaagctcttgaaaacagtcccaaggaacctggtgtacctacaactaagactcctgcagcgactaaacctgaaatgactacaacagctaaagacaagacaacagaaagagacttacgtactacacctgaaactacaactgctgcacctaagatgacaaaagagacagcaactacaacagaaaaaactaccgaatccaaaataacagctacaaccacacaagtaacatctaccacaactcaagataccacaccattcaaaattactactcttaaaacaactactcttgcacccaaagtaactacaacaaaaaagacaattactaccactgagattatgaacaaacctgaagaaacagctaaaccaaaagacagagctactaattctaaagcgacaactcctaaacctcaaaagccaaccaaagcacccaaaaaacccacttctaccaaaaagccaaaaacaatgcctagagtgagaaaaccaaagacgacaccaactccccgcaagatgacatcaacaatgccagaattgaaccctacctcaagaatagcagaagccatgctccaaaccaccaccagacctaaccaaactccaaactccaaactagttgaagtaaatccaaagagtgaagatgcaggtggtgctgaaggagaaacacctcatatgcttctcaggccccatgtgttcatgcctgaagttactcccgacatggattacttaccgagagtacccaatcaaggcattatcatcaatcccatgctttccgatgagaccaatatatgcaatggtaagccagtagatggactgactactttgcgcaatgggacattagttgcattccgaggtcattatttctggatgctaagtccattcagtccaccatctccagctcgcagaattactgaagtttggggtattccttcccccattgatactgtttttactaggtgcaactgtgaaggaaaaactttcttctttaaggattctcagtactggcgttttaccaatgatataaaagatgcagggtaccccaaaccaattttcaaaggatttggaggactaactggacaaatagtggcagcgctttcaacagctaaatataagaactggcctgaatctgtgtattttttcaagagaggtggcagcattcagcagtatatttataaacaggaacctgtacagaagtgccctggaagaaggcctgctctaaattatccagtgtatggagaaatgacacaggttaggagacgtcgctttgaacgtgctataggaccttctcaaacacacaccatcagaattcaatattcacctgccagactggcttatcaagacaaaggtgtccttcataatgaagttaaagtgagtatactgtggagaggacttccaaatgtggttacctcagctatatcactgcccaacatcagaaaacctgacggctatgattactatgccttttctaaagatcaatactataacattgatgtgcctagtagaacagcaagagcaattactactcgttctgggcagaccttatccaaagtctggtacaactgtccttagactgatgagcaaaggaggagtcaactaatgaagaaatgaataataaattttgacactgaaaaacattttattaataaagaatattgacatgagtataccagtttatatataaaaatgtttttaaacttgacaatcattacactaaaacagatttgataatcttattcacagttgttattgtttacagaccatttaattaatatttcctctgtttattcctcctctccctcccattgcatggctcacacctgtaaaagaaaaaagaatcaaattgaatatatcttttaagaattcaaaactagtgtattcacttaccctagttcattataaaaaatatctaggcattgtggatataaaactgttgggtattctacaacttcaatggaaattattacaagcagattaatccctctttttgtgacacaagtacaatctaaaagttatattggaaaacatggaaatattaaaattttacacttttactagctaaaacataatcacaaagctttatcgtgttgtataaaaaaattaacaatataatggcaataggtagagatacaacaaatgaatataacactataacacttcatattttccaaatcttaatttggatttaaggaagaaatcaataaatataaaatataagcacatatttattatatatctaaggtatacaaatctgtctacatgaagtttacagattggtaaatatcacctgctcaacatgtaattatttaataaaactttggaacattaaaaaaataaattggaggcttaaaaaaaaaaaaaaaaa

SEQ ID NO: 3 - protein, 7 aa, Homo sapiens
KEPAPTT

(19) United States
(12) Patent Application Publication
(10) Pub. No.: US 2017/0312335 A1
(43) Pub. Date: Nov. 2, 2017

(54) TOPICAL USE OF PRG4 FOR TREATMENT OF ALLERGY AND SYMPTOMS OF INFLAMMATION

(71) Applicant: Lubris LLC, Framingham, MA (US)

(72) Inventors: Edward R. Truitt, III, San Diego, CA (US); Benjamin Sullivan, Boston, MA (US)

(21) Appl. No.: 15/396,391

(22) Filed: Dec. 30, 2016

Related U.S. Application Data
(60) Provisional application No. 62/273,059, filed on Dec. 30, 2015.

(51) Int. Cl. A61K 38/17; A61K 45/06; A61K 9/00; A61M 15/08; A61K 9/08

(52) U.S. Cl. CPC A61K 38/1709; A61M 15/08; A61K 45/06; A61K 9/08; A61K 9/0014; A61K 9/0048; A61K 9/0043; A61K 9/0053

(57) ABSTRACT

Disclosed are methods involving the therapeutic use of human PRG4 (PRG4) protein, to ameliorate the symptoms associated with allergies and/or respiratory infections.

### CROSS-REFERENCE TO RELATED APPLICATIONS

This application claims priority to and the benefit of U.S. Provisional Patent Application No. 62/273,059 filed Dec. 30, 2015, the entire contents of which is incorporated by reference herein,

### FIELD OF THE INVENTION

This invention relates to new therapies designed to treat allergies and/or respiratory infections. More particularly, it relates to PRG4-containing pharmaceutical compositions and their use for ameliorating the symptoms associated with allergies and/or respiratory infections and/or inflammation. Such symptoms include congestion, post-nasal drip, coughing, sneezing, runny nose, itchy throat and itchy, watery eyes.

### BACKGROUND OF THE INVENTION

The proteoglycan 4 (PRG4) gene encodes a highly glycosylated protein now known as PRG4. PRG4 originally was identified as an expression product of synovial fibroblasts and shown to possess boundary lubricating ability. O-linked β(1-3) Gal-GalNAc oligosaccharides pendant from a large central mucin-like domain of 940 amino acids encoded by exon 6 of the PRG4 gene subsequently were shown to be critical to the molecule's boundary lubricating ability. The function of PRG4 heretofore has been, for example, associated with providing lubrication of interfacing tissues and treatment of visual impairments.

A large and increasing proportion of the population suffer from allergies and/or respiratory infections. Allergy is a species of inflammation, an adaptive immune reaction that includes such maladies as allergic asthma, atopic dermatitis, allergic rhinitis and several ocular allergic diseases. Allergic Rhinitis occurs when the body's immune system over-responds to specific, non-infectious particles such as plant pollens, molds, dust mites, animal hair, industrial chemicals, foods, medicines, and insect venom. During an allergic attack, antibodies, primarily immunoglobin E (IgE), attach to mast cells (cells that release histamine) in the skin and mucous membranes. Once IgE connects with the mast cells, a number of chemicals are released. One of the chemicals, histamine, opens the blood vessels and causes skin redness and swollen membranes. When this occurs in the nose, sneezing and congestion are the result. Signs and symptoms include a runny or stuffy nose, sneezing, red, itchy, and watery eyes, and swelling around the eyes. In addition, similar signs and symptoms are found to be associated with respiratory infections, e.g., upper respiratory infections. There exists a long-felt need for effective means to treat such symptoms.

### SUMMARY OF THE INVENTION

As disclosed herein, it has been discovered that PRG4 is useful for alleviating the symptoms associated with allergies and/or respiratory infections, and/or dermatitis, and may also facilitate the body's mechanisms for improved removal from body surfaces of allergens and cellular and matrix debris which trigger chronic inflammation. It has now been discovered that PRG4 can reduce the symptoms associated with allergies and/or respiratory infections and extends and exploits PRG4's function beyond its well-known lubricating properties. Such symptoms include congestion, post-nasal drip, coughing, sneezing, runny nose, itchy throat, itchy skin, and itchy watery eyes, to name a few. PRG4 can therefore be used in a number of novel ways in therapeutic contexts to reduce the symptoms associated with allergies and/or respiratory infections and may inhibit development of chronic inflammation which begins as an inflammatory event natural to a healing process. As used herein, upper respiratory tract, refers to the parts of the respiratory system lying above the sternal angle (outside of the thorax), above the glottis (vocal cords), or above the cricoid cartilage and includes the nose, sinuses, nasal passages, and nasopharynx. The respiratory tract includes the upper respiratory tract, as well as the lungs, and particularly the bronchial and tracheal trees.

The present invention provides, in various embodiments, compositions, and methods of use thereof, for managing one or more allergy and/or upper respiratory infection symptoms. In an embodiment of the invention, a method is provided of treating a patient exhibiting such symptoms or at risk of developing such symptoms including congestion, post-nasal drip, coughing, wheezing, sneezing, runny nose, itchy skin, itchy throat, itchy eyes, and watery eyes, the method including the step of administering to a surface of the patient's body suffering or at risk of developing allergy symptoms, e.g., eyes, skin, or respiratory tract, e.g., upper respiratory tract, an amount of a PRG4-containing composition sufficient to ameliorate the symptoms. More specifically, a method is provided of treating an allergy and/or respiratory infection, the method comprising applying, typically directly, to a body surface such as the eyes or respiratory tract a composition containing PRG4 in an amount sufficient to treat the respiratory symptom.

Accordingly, the present invention provides for PRG4 use in the treatment of an allergy and/or respiratory infection and for use in treating one or more symptoms thereof. In various embodiments, such uses include, by way of non-limiting example, use of PRG4-containing pharmaceutical compositions for treatment of allergy and/or upper respiratory infection symptoms. Such pharmaceutical compositions include those suitable for topical administration to oral, nasal, pulmonary or ocular tissues, or to skin. The compositions may conveniently be presented in unit dosage form and may be prepared by any method well known in the art of pharmacy. The amount of PRG4 which can be combined with a carrier material to produce a single dosage form will vary depending upon the patient being treated and the particular mode of administration.

In certain instances, the beneficial effects of PRG4-containing nasal compositions are achieved by topical intra-nasal administration of PRG4 to the mucosal surface of the nose and sinuses. The method of the invention comprises the step of depositing intranasally onto the mucosal surface of the nose and sinuses of a patient an amount of a nasal composition comprising PRG4 in an amount sufficient to ameliorate at least one allergy and/or upper respiratory infection symptom. In an embodiment of the invention, the PRG4-containing nasal composition is administered as a nasal spray. Such nasal sprays may be contained within a nasal spray bottle wherein actuation of the spray bottle ejects a plume of the pharmaceutical composition in an amount sufficient to reduce at least one nasal-associated symptom. In another embodiment, the composition is administered as an inhalable aerosol or the like for treatment of the bronchial or tracheal tree or the lungs.

In certain instances, as provided herein, the beneficial anti-inflammatory effects of PRG4-containing compositions are achieved by administration of PRG4 to the surface of the eye. The method of the invention comprises the step of depositing onto the surface of one or both eyes of a patient an amount of an ophthalmically acceptable solution comprising PRG4 in an amount sufficient to alleviate at least one eye-associated allergy and/or upper respiratory infection symptom. Such symptoms include, for example, red, watery and/or itchy eyes. In an embodiment of the invention, the PRG4-containing ocular composition is administered as eye drops. In other instances, application of a PRG4-containing ocular composition is administered to the eye as a delivery method to the nasal mucosa, e.g., after excess drop passes through the nasolacrimal duct into the nose.

In yet other instances, the beneficial effects of PRG4-containing oral compositions are achieved by topical administration of PRG4 to the mucosal surface of the oral cavity including the oral or nasopharyngeal airways. The method of the invention comprises the step of depositing onto the mucosal surface of the oral cavity in a patient an amount of an orally acceptable composition comprising PRG4 in an amount sufficient to alleviate at least one oral associated allergy symptom. Such symptoms include, for example, coughing and/or itchy swollen mouth. In an embodiment of the invention, the PRG4-containing oral composition is administered as an aqueous solution, gel or paste. The composition may also be prepared as a gum or lozenge.

In one aspect, the method is practiced on patients who are not suffering from an oral cavity lubrication deficiency, xerostomia, or oral ulcerations. In another aspect, the method is practiced on patients who are not suffering from dry eye disease, vision impairments or vision aberrations.

### BRIEF DESCRIPTION OF THE INVENTION

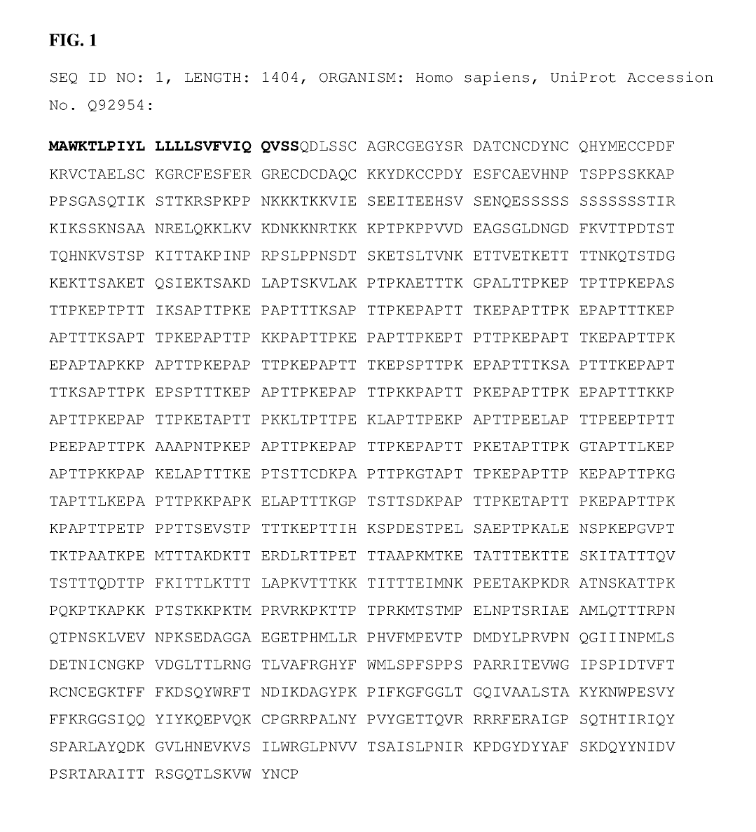

FIG. 1 is the full length PRG4 amino acid sequence, showing the secretion signal (first 24 amino acids which are removed during post translational processing).

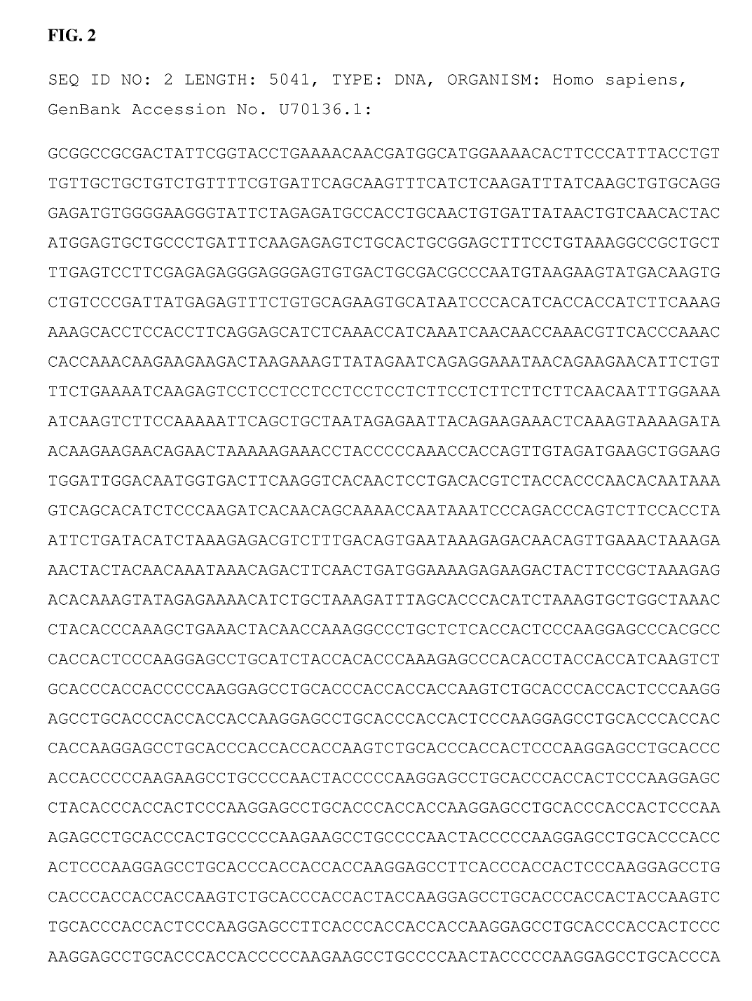

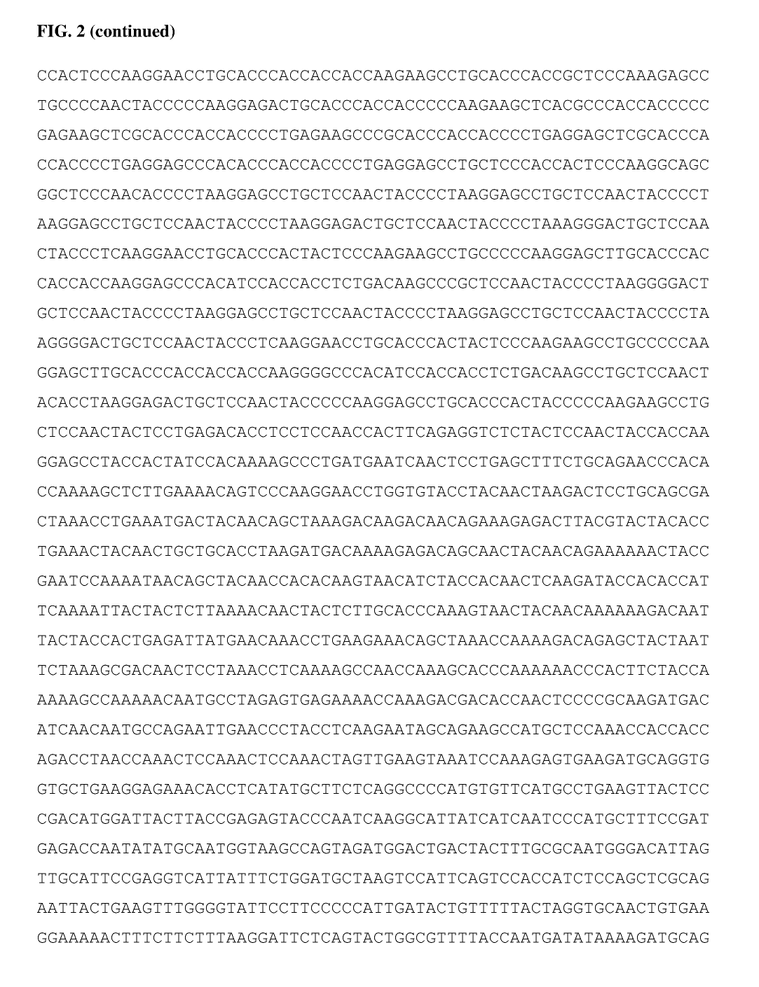

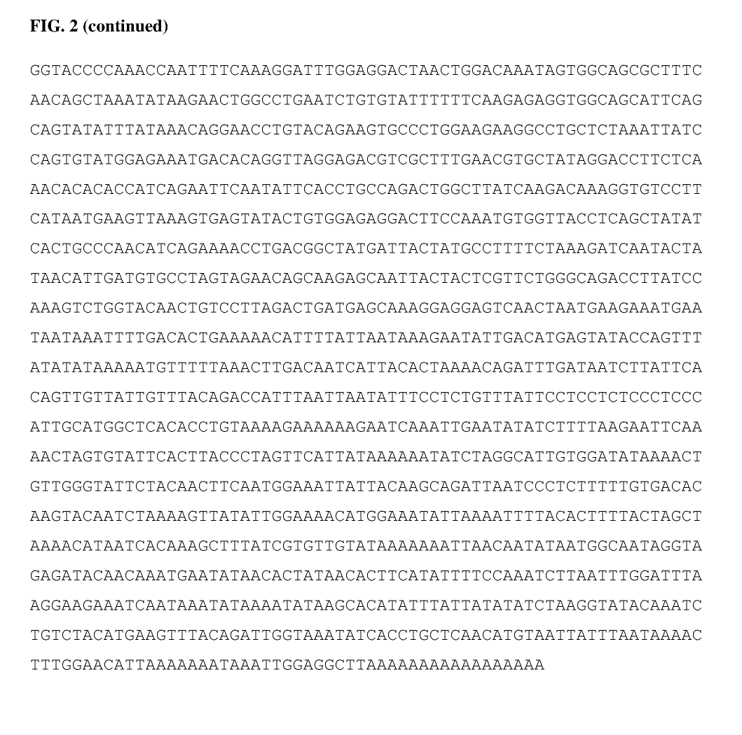

FIG. 2 is the full length human DNA encoding PRG4.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Allergy is a species of inflammation, an adaptive immune reaction that includes such maladies as allergic asthma, atopic dermatitis, allergic rhinitis and several ocular allergic diseases. It is characterized by a Th2 mediated humoral response to antigenic challenge. In a typical Type I hypersensitivity allergic reaction, initial exposure to an allergen causes B cells to produce IgE antibodies that bind to the surface of mast cells/basophils, sensitizing those cells to the allergen. Subsequent exposure to the same antigen results in an immediate degranulation of the mast cell and subsequent release of histamine, prostaglandins, leukotrienes (LTC4, LTD4, LTE4), chemokines (CXCL8, CXCL10, CCL2, CCL4, CCL5), proteases (tryptase, chymase) and cytokines such as IL-4, IL-5, and IL-13 (Janeway et al., Immunobiology: The Immune System in Health and Disease. 5th edition. New York: Garland Science 2001; Larché et al., Nat Rev Immunol. 2006; 6(10):761-71). These effector molecules cause dilation of small blood vessels, increased vascular permeability, bulk mucus production, and local contraction of smooth muscles resulting in the familiar symptoms associated with allergic reactions. After several hours, the late phase of the allergic reaction sees the recruitment of eosinophils, basophils and Th2 lymphocytes to the site of the reaction. Eosinophils release a series of granule proteins such as eosinophil cationic protein, major basic protein, eosinophil peroxidase and eosinophil-derived neurotoxin, as well as a series of reactive oxygen species (peroxides) that act to clean out the area through oxidative stress and ribonuclease activity. While toxic to invading organisms, eosinophil responses also disrupt host cells in the vicinity of the allergic reaction.

Once the positive feedback loop of tissue damage and inflammatory cell recruitment has been established, a chronic inflammatory state may persist, even without sustained exposure to the original allergen (Murdoch J R, Lloyd C M. Chronic Inflammation and Asthma. Mutat Res. 2010; Aug. 7; 690(1-2):24-39. doi: 10.1016/j.mrfmmm.2009.09.005. Epub 2009 Sep. 19). In particular, chronic inflammation is accompanied by remodeling of the tissues that result in compromised epithelial barrier function, matrix metalloproteinase expression and mucus gland hyperplasia, as well as TGF-β mediated fibrosis (Murdoch et al.). For instance, in chronic asthma, repeated cycles of eosinophil mediated damage and subsequent matrix synthesis by fibroblasts leads to thickened, constricted, less elastic airways, with airway remodeling being linked directly to the chronicity of the disorder (Murdoch et al.). It is noteworthy that the dominant asthma therapies aimed at reducing inflammation (corticosteroids), exhibit limited efficacy in ameliorating remodeling (Murdoch et al.; Ward C, Walters H., Curr Opin Allergy Clin Immunol. 2006; February; 5(1):43-8).

Without wishing to be bound by theory, the current invention is based in part on the recognition that the sequelae associated with an immune dysregulation or chronic exposure to allergens may result in impaired mechanical clearance of antigens, PAMPs and DAMPs, as well as compromised tissue function associated with repeated remodeling. Not only do these processes potentiate inflammation, but also result in long-term damage to the tissues, whether respiratory, ocular, or the skin, and it is believed the positive feedback loop conditions are similar.

Accordingly, in an embodiment of the invention, the application of PRG4-containing compositions to tissues undergoing a chronic allergic response will benefit from improved allergen clearance as well as a mediated fibrotic response. The boundary lubricating ability of such compositions will also prevent mucus particulate adhesion to the epithelium, as well as improved hydration, as the highly charged PRG4 molecule is hygroscopic and will retain water along the interface of the epithelia. Due to the improved tissue surface lubrication following the application of PRG4-containing compositions, mechanical clearance of allergens will require less force as the friction between particulate (comprised of bulk mucin, debris and allergens) and the epithelium is reduced. With lower friction, mechanical clearance through, e.g. blinking (eye), or air flow and mucociliary clearance (respiratory system), will require less force and result in less tissue damage and inflammation. Administration of PRG4-containing compositions to patients suffering from chronic allergy will also result in mitigation of fibrosis through prevention of fibroblast adhesion and migration which will reduce the overall fibrotic response.

More specifically, the present invention is based on the discovery that PRG4 can be used to treat the symptoms associated with allergies and/or respiratory infections. As used herein, “treat” may involve preventing the worsening of symptoms or may involve alleviating, ameliorating, reducing, lessening or inhibiting the symptoms associated with allergies and/or respiratory infections in a patient. Such symptoms include, for example, congestion, post-nasal drip, coughing, wheezing, sneezing, runny nose, itchy throat and itchy watery eyes, to name a few. PRG4 can therefore be used in a number of novel ways in therapeutic contexts to reduce allergy and/or upper respiratory infection symptoms. The invention provides, in various embodiments, compositions, and methods of use thereof, for managing such symptoms.

In various embodiments, such uses include, by way of non-limiting example, use of PRG4-containing pharmaceutical compositions for treatment of the symptoms. As disclosed in detail below, such compositions of the present invention include those suitable for oral, intra-tracheal, intranasal, or ocular administration. The formulations may conveniently be presented in unit dosage form and may be prepared by any methods well known in the art of pharmacy. The amount of PRG4 which can be combined with a carrier material to produce a single dosage form will vary depending upon the patient being treated and the particular mode of administration.

### PRG4 (Lubricin)

PRG4, also referred to as lubricin, is a lubricating polypeptide, which in humans is expressed from the megakaryocyte stimulating factor (MSF) gene, also known as PRG4 (see NCBI Accession Number AK131434-U70136). Lubricin is a ubiquitous, endogenous glycoprotein that coats the articulating surfaces of the body. Lubricin is highly surface active molecule (e.g., holds onto water), that acts primarily as a potent cytoprotective, anti-adhesive and boundary lubricant. It is characterized by a long, central mucin-like domain located between terminal protein domains that allow the molecule to adhere and protect tissue surfaces. Its natural form, in all mammals investigated, contains multiple repeats of an amino acid sequence which is at least 50% identical to KEPAPTT. Natural lubricin typically comprises multiple redundant forms of this repeat, but typically includes proline and threonine residues, with at least one threonine being glycosylated in most repeats. The threonine anchored O-linked sugar side chains are critical for lubricin's boundary lubricating function. The side chain moiety typically is a β(1-3)Gal-GalNAc moiety, with the β(1-3)Gal-GalNAc typically capped with sialic acid or N-acetylneuraminic acid. The polypeptide also contains N-linked oligosaccharides. The gene encoding naturally-occurring full length lubricin contains 12 exons, and the naturally-occurring MSF gene product contains 1,404 amino acids (including the secretion sequence) with multiple polypeptide sequence homologies to vitronectin including hemopexin-like and somatomedin-like regions. Centrally-located exon 6 contains 940 residues. Exon 6 encodes the repeat rich, 0-glycosylated mucin domain.

The amino acid sequence of the protein backbone of lubricin may differ depending on alternative splicing of exons of the human MSF gene. This robustness against heterogeneity was exemplified when researchers created a recombinant form of lubricin missing 474 amino acids from the central mucin domain, yet still achieved reasonable, although muted, lubrication (Flannery et al., Arthritis Rheum 2009; 60(3):840-7). PRG4 has been shown to exist not only as a monomer but also as a dimer and multimer disulfide-bonded through the conserved cysteine-rich domains at both N- and C-termini. Lubris, LLC has developed a full-length recombinant form of human lubricin. The molecule is expressed using the Selexis Chinese hamster ovary cell line (CHO-M), with a final apparent molecular weight of 450-600 kDa, with polydisperse multimers frequently measuring at 2,000 kDa or more, all as estimated by comparison to molecular weight standards on SDS tris-acetate 3-8% polyacrylamide gels. Of the total glycosylations, about half comprise two sugar units (GalNAc-Gal), and half three sugar units (GalNAc-Gal-Sialic acid). This method of recombinant human PRG4 production is provided in International Patent Application No. PCT/US014/061827.

Any one or more of various native and recombinant PRG4 proteins and isoforms may be utilized in the various embodiments described herein. For instance, U.S. Pat. Nos. 6,433,142; 6,743,774; 6,960,562; 7,030,223, and 7,361,738 disclose how to make various forms of human PRG4 expression product, each of which is incorporated herein by reference. Preferred for use in the practice of the invention is full length, glycosylated, recombinant PRG4, or lubricin, expressed from CHO cells. This protein comprises the 1,404 amino acid sequence of FIG. 1 and is encoded for by the nucleotide sequence of FIG. 2. The protein includes a central exon comprising repeats of the sequence KEPAPTT variously glycosylated with O-linked β (1-3) Gal-GalNAc oligosaccharides, and including N and C-terminal sequences with homology to vitronectin. The molecule is polydisperse with the glycosylation pattern of individual molecules varying, and can comprise monomeric, dimeric, and multimeric species.

As used herein, the term “PRG4” is used interchangeably with the term “lubricin.” Broadly, these terms refer to any functional isolated or purified native or recombinant properly glycosylated PRG4 proteins, homologs, functional fragments, isoforms, and/or mutants thereof. In one embodiment, the invention includes homologs, functional fragments, isoforms, and/or mutants of PRG4 that have at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity with the full length PRG4 protein sequence of SEQ ID NO:1 (FIG. 1). All useful molecules include the sequence encoded by exon 6, or homologs or truncated versions thereof, for example, versions with fewer repeats within this central mucin-like KEPAPTT-repeat domain, together with O-linked glycosylation. In one embodiment, the invention includes homologs, functional fragments, isoforms, and/or mutants of PRG4 that have at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity with the amino acid sequence or nucleic acid sequence corresponding to exon 6 of PRG4. All useful molecules also include at least the biological active portions of the sequences encoded by exons 1-5 and 7-12, i.e., sequences responsible for imparting to the molecule its affinity for ECM and endothelial surfaces. In certain embodiments, a preferred PRG4 protein has an average molar mass of between 50 kDa and 500 kDa, preferably between 224 to 467 kDa, comprising one or more biological active portions of the PRG4 protein, or functional fragments, such as a lubricating fragment, or a homolog thereof. In a more preferred embodiment, a PRG4 protein comprises monomers of average molar mass of between 220 kDa to about 280 kDa.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino acid or nucleic acid sequence). The percent identity between the two sequences is a function of the number of identical positions shared by the sequences (i.e., % homology=(# of identical positions/total # of positions)times 100). The determination of percent homology between two sequences can be accomplished using a mathematical algorithm. A non-limiting example of a mathematical algorithm utilized for the comparison of two sequences is the algorithm of Karlin and Altschul, (1990) Proc. Natl. Acad. Sci. USA, 87:2264-68, modified as in Karlin and Altschul, (1993) Proc. Natl. Acad. Sci. USA, 90:5873-77. Such an algorithm is incorporated into the NBLAST and XBLAST programs of Altschul et al., (1990) J. Mol. Biol., 215:403-10. BLAST nucleotide searches can be performed with the NBLAST program, score=100, wordlength=12. BLAST protein searches can be performed with the XBLAST program, score=50, wordlength=3. To obtain gapped alignments for comparison purposes, Gapped BLAST can be utilized as described in Altschul et al., (1997) Nucleic Acids Research, 25(17):3389-3402. When utilizing BLAST and Gapped BLAST programs, the default parameters of the respective programs (e.g., XBLAST and NBLAST) can be used.

Methods for isolation, purification, and recombinant expression of a PRG4 protein are well known in the art. In certain embodiments, the method starts with cloning and isolating mRNA and cDNA encoding PRG4 proteins or isoforms using standard molecular biology techniques, such as PCR or RT-PCR. The isolated cDNA encoding the PRG4 protein or isoform is then cloned into an expression vector, and expressed in a host cell for producing recombinant PRG4 protein, and isolated from the cell culture supernatant. A method for production of recombinant human PRG4 is provided in International Patent Application No. PCT/US014/061827.

### PRG4-Containing Compositions

PRG4 containing compositions of the present invention include those suitable for intranasal, intra-tracheal, ocular, topical, or oral administration. Such compositions are designed for application to the surfaces of the respiratory tract, or eyes, or skin, to relieve the symptoms associated with allergies and/or infections. The compositions may conveniently be presented in unit dosage form and may be prepared by any methods well known in the art of pharmacy. In addition to PRG4, the pharmaceutical compositions of the present invention may further include one or more pharmaceutically acceptable carriers or vehicles including any acceptable materials, and/or any one or more additives known in the art. As used herein, the term “carriers” or “vehicle” refer to carrier materials suitable for intranasal, intra-tracheal, intra-bronchial, ocular, topical, or oral drug administration. Carriers and vehicles useful herein include any such materials known in the art, which are nontoxic and do not interact with other components of the composition in a deleterious manner. Various additives, known to those skilled in the art, also may be included in the composition.

The amount of PRG4 which can be combined with a carrier material to produce the compositions of the invention will generally be that amount of the compound which produces a therapeutic effect, i.e., reduction in allergy and/or upper respiratory infection symptoms. The concentration of PRG4 may vary widely, from a few micrograms per milliliter to as many as 200 or 300 micrograms per milliliter or more.

Methods of preparing these compositions include the step of bringing into association PRG4 with the carrier and, optionally, one or more accessory ingredients. In general, the formulations are prepared by uniformly and intimately bringing into association a PRG4 with liquid carriers, or solid carriers, or both. The compositions may also contain adjuvants such as preservatives, wetting agents, emulsifying agents and dispersing agents.

In certain embodiments, the PRG4-containing compositions of the invention may include one or more additional therapeutic agents designed to reduce the symptoms of allergies and/or infections. Such therapeutic agents include antihistamines, corticosteroids, anti-inflammatories, decongestants and mast cell stabilizers. Exemplary antihistamines include azelastine hydrochloride and olopatadine, to name a few. Corticosteroids such as fluticasone propionante, fluticasone furate, triamcinolone, flunisolide, mometasone and beclomethasone may be used in the compositions. Examples of mast cell stabilizers include ketotifen fumarate, pemirolast potassium, nedocromil sodium, lodoxamide and cromolyn. Decongestants optionally included for use in the nasal compositions of the invention include, for example, naphazoline HCL phenylephrine HCL. tetrahydrozoline HCL, and/or oxymetazoline HCL.

In a specific embodiment of the invention, compositions for alleviating the nasal symptoms associated with allergies and/or upper respiratory infections are provided. In an aspect of the invention, said symptoms are selected from the group consisting of congestion, post-nasal drip, coughing, sneezing, runny nose, itchy throat, itchy eyes, and watery eyes.

PRG4 can be conveniently administered nasally to patients exhibiting the symptoms of allergies and/or upper respiratory infections by formulating it into a nasal dosage form comprising a therapeutically effective concentration of PRG4 and a pharmaceutically acceptable nasal carrier. Suitable non-toxic, non-irritating, pharmaceutically acceptable nasal carriers will be apparent to those skilled in the art of nasal pharmaceutical formulations. Examples of pharmaceutically acceptable nasal carriers include water; physiological saline solution; alcohols, such as ethanol and isopropanol; glycols, such as propylene glycol; glycol ethers, such as polyethylene glycols. Additional ingredients, such as buffers, preservatives, osmotic agents, gelling agents, wetting agents, may also be present.

PRG4 can be formulated into a nasal solution for use as drops or as a spray, a nasal suspension, a nasal ointment, or a nasal gel. The preferred nasal dosage form is a solution which is applied as drops or an aerosol spray. When a nasal dosage form of PRG4 is applied as an aerosol spray, a propellant gas may be added to the active ingredient and carrier composition. In a specific embodiment of the invention, a nasal composition comprising PRG4 in an amount sufficient to alleviate at least one allergy and/or upper respiratory infection symptom is provided.

For nasal administration, PRG4 is dissolved or suspended in solutions or mixtures of excipients (e.g., preservatives, viscosity modifiers, emulsifiers, buffering agents, etc.) in a pressurized, or non-pressurized, dispenser that delivers a specifically controlled amount of spray containing a metered dose into one or both nostrils. The dose typically is metered by the spray pump, which is typically finger or hand actuated. It may be designed to administer the intended dose with multiple sprays, e.g., two sprays, e.g., one in each nostril, or as a single spray, or to vary the dose in accordance with the weight, sex, or maturity of the patient.

In one aspect of the invention, a nasal spray bottle comprising a spray nozzle and containing a pharmaceutical composition comprising PRG4 is provided. The nasal spray bottle may be one wherein actuation of the nasal spray nozzle ejects a plume of the pharmaceutical composition comprising an amount of PRG4 sufficient to ameliorate at least one symptom associated with allergies and/or upper respiratory infections. Such symptoms include those selected from the group consisting of congestion, post-nasal drip, coughing, sneezing, runny nose, itchy throat, itchy eyes, and watery eyes.

The present invention also provides a method for ameliorating the ocular symptoms associated with an allergic reaction, or upper respiratory infection, including, for example, itchy, red and watery eyes. According to this method, application to the eye surface of a sufficient amount of PRG4 has the effect of ameliorating the symptoms. These effects include diminishment of eye itchiness, redness and/or wateriness. These beneficial effects are achieved in appropriate patients suffering from allergies and/or upper respiratory infections simply by depositing on the surface of the eye enough PRG4 to ameliorate the symptoms.

In an embodiment of the invention, the ocular composition is formulated as an eye drop. In such an instance, the PRG4 can be deposited in the eye as a drop of solution having a volume of 10 to 100 microliters. In another embodiment, the PRG4 can be deposited in the eye as a drop of solution having a volume of 15 to 30 microliters. Eye drops suitable for topical application to an ocular surface comprise a therapeutically effective concentration of a PRG4 protein disposed in an ophthalmically acceptable balanced salt solution, e.g., phosphate buffered saline. Ophthalmically acceptable compositions are considered suitable for topical application to the ocular surface if, upon application, they lack unacceptable eye toxicity, burning, itchiness, viscosity, etc. The concentration of PRG4 may vary widely, from a few micrograms per milliliter to as many as 200 or 300 micrograms per milliliter or more. More dilute solutions may permit the patient to titrate the therapeutic dose to suit his or her allergy and/or upper respiratory infection by adding multiple drops.

In certain embodiments, the eye drops used in the present invention also may comprise one or more optional ingredients such as a therapeutically effective concentration of sodium hyaluronate, hyaluronic acid, and/or phospholipid. Exemplary phospholipids include L-α-dipalmitoylphosphatidylcholine, phosphatidylcholine, phosphatidylethanolamine and sphingomyelin.

The PRG4 typically is dissolved in an ophthalmically acceptable balanced salt solution comprising at least three electrolytes, including, for example, sodium chloride (NaCl) 0.64%, potassium chloride (KCl) 0.075%, calcium chloride dihydrate (CaCl2.2H2O) 0.048%, magnesium chloride hexahydrate (MgCl2.6H2O) 0.03%, sodium acetate trihydrate (C2H3NaO2.3H2O) 0.39%, sodium citrate dihydrate (C6H5Na3O7.2H2O) 0.17%, and sodium hydroxide and/or hydrochloric acid (to adjust pH to approximately 7.5) with an osmolarity of approximately 300 mOsms/L. In other embodiments, the aqueous vehicle may comprises 128 mM sodium, 24 mM potassium, approximately 113 mM chloride, approximately 0.4 mM calcium, approximately 0.3 mM magnesium, approximately 5 mM HCO3-, approximately 1 mM citrate, approximately 14 mM phosphate, approximately 15 mM acetate, and sodium hydroxide and/or hydrochloric acid sufficient to adjust pH to approximately 7.5, and with an osmolarity of approximately 200-300 mOsm/L.

In certain embodiments, the present invention provides a pharmaceutical composition suitable for topical application to an ocular surface comprising a therapeutically effective concentration of PRG4 protein suspended in an ophthalmically acceptable balanced salt solution, comprised of sodium (Na+) of approximately 128 mM, potassium (K+) of approximately 24 mM, chloride (Cl—) of approximately 113 mM, calcium (Ca2+) of approximately 0.4 mM, magnesium (Mg2+) of approximately 0.3 mM, HCO3- of approximately 5 mM, citrate of approximately 1 mM, phosphate of approximately 14 mM, acetate of approximately 15 mM, and sodium hydroxide and/or hydrochloric acid (to adjust pH to approximately 7.5) with an osmolarity of approximately 300 mOsms/L.

In certain embodiments, the pharmaceutical composition of the present invention is prepared in a pharmaceutically acceptable carrier, such as a phosphate buffered saline or an osmotically balanced salt solution of tear electrolytes, including one or more of sodium chloride in about 44% to about 54% mole fraction, potassium chloride in about 8% to about 14% mole fraction, sodium bicarbonate in about 8% to about 18% mole fraction, potassium bicarbonate in about 0% to about 4% mole fraction, calcium chloride in about 0% to about 4% mole fraction, magnesium chloride in about 0% to about 4% mole fraction, trisodium citrate in about 0% to about 4% mole fraction, and hydrochloric acid in about 0% to about 20% mole fraction or sodium hydroxide in about 0% to about 20% mole fraction. In certain embodiments, the pharmaceutical carrier can be formulated to generate an aqueous electrolyte solution in about 150-200 mM range. Other suitable formulations, such as ointments, creams, gels, pastes, and the like, suitable for ocular administration, are also contemplated in the present invention. In certain embodiments, electrolytes provide proper osmotic balance when combined with PRG4 to make a solution ophthalmically acceptable.

In another embodiment of the invention, compositions are provided for alleviating the oral symptoms associated with allergies and/or respiratory infections. In an aspect of the invention, said symptoms are selected from the group consisting of congestion, post-nasal drip, coughing, wheezing, sneezing, runny nose, itchy throat. The orally administered compositions are preferably formulated as an aqueous solution, gel or paste and administered via a mouth spray, mouthwash, toothpaste, rinse or gel. The composition may also be prepared as a gum or lozenge.

PRG4 can be conveniently administered orally to patients exhibiting the symptoms of allergies and/or respiratory infections by formulating it into an oral dosage form comprising a therapeutically effective concentration of PRG4 and a pharmaceutically acceptable oral carrier. In certain embodiments, an orally acceptable composition (e.g., an oral care product described herein) comprises a demulcent, an astringent, an emollient, a sweetener, a stimulator, or combinations thereof. Suitable sweeteners may be readily selected, and the amount of sweetener incorporated into the present composition will be determined by taste. Generally, the sweetener may be any compound or compounds that cause sweetness or intensify sweetness. Examples of suitable sweeteners are set forth in the Encyclopedia of Chemical Technology, vol. 19, 2d Ed., New York: John Wiley & Sons, 1969, at pp. 593-607.

It is optional that the PRG4 oral compositions contain one or more preservatives, typically an anti-oxidant present in an amount effective to retard oxidation and/or inactivation of the fluid extract. As with sweeteners, the selection of a preservative or preservatives will be readily made by one skilled in the art. Examples of suitable preservatives include ascorbyl palmitate, butylated hydroxyanisole, butylated hydroxytoluene, potassium or sodium sorbate, sodium bisulfite, sodium metabisulfite, sorbic acid, sulfur dioxide, and sodium or potassium benzoate.

Other components which may, if desired, be incorporated into the present oral compositions include coloring agents, which may be either natural or synthetic, flavoring agents, flavor preserving agents, diluting agents, emulsifying agents, excipients, pH buffering agents, and the like. Reference may be had to the Kirk-Othmer Encyclopedia of Chemical Technology, 3rd Ed., in Volume 6, for F.D.& C. colorants and corresponding chemical structures.

Flavorings are also optional, as incorporation of citric and/or ascorbic acids into the composition will in the absence of any additional flavoring agents provide a pleasant, citrus flavor. Additional flavorings may include other natural or artificial flavors, e.g., mint oils such as peppermint, wintergreen (methyl salicylate), spearmint, eucalyptus, etc., citrus oils such as lemon oil, orange oil, lime oil, grapefruit oil, fruit essences such as apple essence, peach essence, raspberry essence, and the like.

### Methods of Treatment

The present invention provides, in various embodiments, methods for managing one or more allergy and/or respiratory infection symptoms. The present invention is based on the discovery that PRG4 when applied topically to a tissue surface can be used to treat the symptoms associated with allergies and/or respiratory infections. As used herein, “treat” may involve preventing the worsening of symptoms or may involve alleviating, ameliorating, reducing, lessening or inhibiting the symptoms associated with allergies and/or upper respiratory infections in a patient. PRG4 can therefore be used in a number of novel ways in therapeutic contexts to reduce allergy and/or upper respiratory infection symptoms.

In an embodiment of the invention, a method is provided of treating a patient exhibiting symptoms associated with allergies and/or respiratory infections the method comprising the step of administering topically to a surface of the respiratory tract, or eyes, of the patient an amount of a composition comprising PRG4 sufficient to ameliorate the symptoms.

Accordingly, the present invention provides for PRG4 use in the treatment of an allergy and/or upper respiratory infection and for use in treating one or more symptoms selected from the group consisting of congestion, post-nasal drip, coughing, sneezing, runny nose, itchy throat, itchy eyes and watery eyes. The treatment according to the invention comprises topically administering a composition comprising PRG4 in an amount sufficient to treat the symptoms of the allergy and/or infection. In a specific embodiment, the composition has a concentration of PRG4 within the range of 5 μg/mL and 5,000 μg/mL. In another embodiment of the invention, the composition has concentration of PRG4 within the range of 10 μg/mL and 300 μg/mL. In yet another embodiment, the concentration of PGR4 is within the range of 50 μg/mL and 200 μg/mL.

In a specific embodiment, the present invention provides a method for treating the nasal symptoms associated with allergy and/or upper respiratory infection. The method of the invention comprises the step of depositing onto the mucosal surface of the nose of a patient an amount of a nasal composition comprising PRG4 in an amount sufficient to ameliorate at least one nasal associated allergy and/or upper respiratory infection symptom. Accordingly, the effects of treatment include, for example, diminishment of post-nasal drip, sneezing, runny nose, congestion and coughing.

In an embodiment of the invention, a PRG4-containing nasal composition is administered as a nasal spray. Such nasal sprays may be contained within a nasal spray bottle wherein actuation of the spray bottle ejects a plume of the pharmaceutical composition in an amount to reduce at least one nasal associated allergy or upper respiratory infection symptom.

In a specific embodiment, the present invention provides a method for treating the ocular symptoms associated with allergy and/or upper respiratory infection. The method of the invention comprises the step of depositing onto the ocular surface of a patient an amount of an ophthalmically acceptable solution containing PRG4 in an amount sufficient to ameliorate at least one ocular associated allergy and/or upper respiratory infection symptom. Such symptoms include, for example, red, watery and/or itchy eyes. Accordingly, the effects of treatment include, for example, diminishment of red, watery and/or itchy eyes.

In one aspect of the invention, the ocular composition is administered as an eye drop. In such an instance, the PRG4 can be deposited in the eye as a drop of solution having a volume of 10 to 100 microliters. In another embodiment, the PRG4 can be deposited in the eye as a drop of solution having a volume of 15 to 30 microliters. Other suitable means for ocular administration, include depositing PGR4 in the eye as an ointment, cream, gel, paste, and the like, suitable for ocular administration, and are also contemplated in the present invention.

In yet another specific embodiment, the present invention provides a method for treating the oral symptoms associated with allergy and/or upper respiratory infection. The method of the invention comprises the step of depositing onto the oral mucosal surface, including the oral or nasopharyngeal airways, of a patient an amount of an orally acceptable solution containing PRG4 in an amount sufficient to ameliorate at least one oral associated allergy and/or upper respiratory infection symptom. Such symptoms include, for example, congestion, post-nasal drip, sneezing, runny nose and itch throat. Accordingly, the effects of treatment include, for example, diminishment of congestion, post-nasal drip, sneezing, runny nose and itch throat.

In an embodiment of the invention, the oral composition is administered as an aqueous composition (or the equivalent in gum or lozenge form) given orally once or 2-6 times per day. For the aqueous composition, it is preferred that the composition be retained in contact with the oral mucosa for a time sufficient to allow coating of the interior of the mouth with the PRG4. Furthermore, the composition may be administered as, for example, a mouthwash, where the mouth is simply rinsed with the aqueous solution, or if desired, the composition may be swallowed. In some embodiments, the oral care pharmaceutical composition comprises PRG4 protein suspended in a aqueous osmotically balanced salt solution, multiphasic emulsification, a gel, liquid, cream, ointment, spray, viscous solution or encapsulated within slow-release devices, or in a lozenge.

In one aspect, the method of the invention is practiced on patients who are not suffering from an oral cavity lubrication deficiency, xerostomia, or oral ulcerations. In another aspect, the method is practiced on patients who are not suffering from dry eye disease, vision impairments or aberrations.

Other features and advantages of the invention will be apparent from the following claims. These and many other variations and embodiments of the invention will be apparent to one of skill in the art upon a review of the description and claims.

1. A method of treating a respiratory allergy symptom, the method comprising applying to a surface of a respiratory tract tissue a composition comprising PRG4 in an amount sufficient to treat the respiratory tract allergy.

2. A method of treating a patient suffering from a symptom selected from the group consisting of congestion, post-nasal drip, coughing, wheezing, sneezing, runny nose, itchy throat, itchy skin, itchy eyes, and watery eyes, the method comprising the step of administering topically to a tissue surface exhibiting or at risk of developing the symptoms an amount of a composition comprising PRG4 sufficient to ameliorate the symptom.

3. The method of claim 2, wherein the symptom is selected from the group consisting of congestion, post-nasal drip, coughing, sneezing, runny nose and itchy throat.

4. The method of claim 1, comprising depositing the PRG4 as a composition having a concentration within the range of 5 μg/mL and 5,000 μg/mL.

5. The method of claim 4, comprising depositing the PRG4 as a composition having a concentration within the range of 10 μg/mL and 300 μg/mL.

6. (canceled)

7. The method of claim 1 wherein the composition is administered either intranasally, orally, or by inhalation.

8. (canceled)

9. The method of claim 8, comprising administering a composition to the oral cavity of a patient who is not suffering from an oral cavity lubrication deficiency, xerostomia, or oral ulcerations.

10. The method of claim 1, wherein the composition is administered as an eye drop.

11. The method of claim 10, comprising depositing said composition onto the surface of the eye of a patient who is not suffering with dry eye disease.

12. The method of claim 10, comprising depositing said composition onto the surface of the eye of a patient who is not suffering with vision impairments or aberrations.

13. The method of claim 10, comprising depositing the PRG4 as a drop of solution having a volume of 10 to 100 microliters.

14. The method of claim 2 comprising applying said composition to the skin.

15. The nasal composition of claim 23, wherein the composition is contained within a nasal spray bottle comprising a spray nozzle.

16-22. (canceled)

23. A nasal composition comprising PRG4 in an amount sufficient to alleviate at least one allergy symptom.

24. The nasal composition of claim 23, wherein the symptom is selected from the group consisting of congestion, post-nasal drip, coughing, sneezing, runny nose, itchy throat, itchy eyes, and watery eyes.

25. The nasal composition of claim 24, wherein the symptom is selected from the group consisting of congestion, post-nasal drip, coughing, sneezing, runny nose and itchy throat.

26. The nasal composition of claim 23, further comprising an additional therapeutic agent designed to reduce an allergy symptom.

27. An ocular composition comprising PRG4 in an amount sufficient to alleviate at least one allergy symptom and further comprising an additional therapeutic agent designed to reduce an allergy symptom.

28. An oral composition comprising PRG4 in an amount sufficient to alleviate at least one allergy symptom and further comprising an additional therapeutic agent designed to reduce an allergy symptom.

29. The composition of claim 26, wherein the additional therapeutic agent is selected from the group consisting of antihistamines, anti-inflammatories, decongestants and mast cell stabilizers.

30. (canceled)
